SEQUENCE LISTING (8 sequences)

SEQ ID NO: 1 - protein, 365 aa, Zea mays
MGTGAVAVAAPDRAKQRRAGGHLWKKALLHFSLCFVMGFFTGFAPSSSSSWKSAAATPQHHPPHRPGDRLAASRVAVDARATLSPPAEAADALSAGATVDLGDDEEDAAQRRLLIVVTTTRSGPGERRRRRGELLRLAHTLRLVRPPVVWVVVEPAADAPATAEVLRGTGVMYRHLAFKPEDNFTTADAEAHVQRNAALAHVEKHRLAGVLHFADAAGVYDVGFFEQIRQIEAFGTWPVATMSAGEKKVVVEGPLCSASKVVGWFSRDFNDGTTRSVTYNTEADLNPAGAAGTRAHTIDVSGFAFNSSILWDPERWGRPTSLPDTSQDSIKFVQEVLLEDRAKLKGIPSDCSQIMVWQYSVPSSR

SEQ ID NO: 2 - DNA, 6760 bp, Zea mays
tctcaaccggtcgtaccttttatataggggagatctggacatgtttctagacgagatccaacagactcacacggataggctaggataatcacgcatgagataaatacttgtatccgagttaacctaatcacgaaccgttcggactaccctgcggaccgttcaggccacaaggtgaaacgaaatcgtacgaccgtgcctgtgtgccaaatatgatgctcaacaactgaaagcatgattttttttaaaaaaaactgaagtttatttttaaaacttaaaatccatctcggtaaaaatgaaattggcttgaaatctcattttcggtgacctccaaaaattaaaaaacgaaaactttaaccctgaaatgaacccacgcaataccggatcctattgtgctggaaccggacaaaaggaaaactgtactttataccggactgcatgctaatgctaatctattaaccgcactccactccaccccctcccgccctgtccacaccaaccacgctcggaggctcccctttcattttcacctcgctccggggccaagtcgccagttcaccaccttcacttcacttcgcaccggaccaccggccgtattcattcctcctccctccctcctcgccatacggctgcagcatcttcccctcaccctcctcattaccccgcgtctctccctcaaaactactcattgctctcaccgttccattgcccccgcggccccgccgtacgctcattcccggctagctcctcctccgcacggccatgatcttgagttcttggcattgatgacgttctagtaccgagcgagccagcccctctttaacccaccttaacaataaacacagcctgcccccaccggccacctcttcttcccccgtgcagctcgagctgattcggcgggctctgccccaacatccacccaccttcgtccaatcccgatccaagaccgtgccgtgatccggcagcagcttccgcgcggccatgggcacgggcgcggtggcggtggcggcgccggaccgggccaagcagcggcgggccggcggccacctgtggaagaaggcactgctccacttctccctctgcttcgtcatgggcttcttcacgggcttcgcgccttcctcctcctcctcctggaagtccgccgccgccaccccccagcaccacccgccgcaccgtcccggggaccgcctcgccgcctcccgcgtggccgtcgacgcgcgcgccaccctgtcgccgcccgcggaggccgccgatgcgctctccgccggcgccacggtggacttgggcgacgacgaggaggacgccgcccagcgccgcctcctgatcgtggtcaccaccacgcgctccggccccggggagcgccgccgccgccggggcgagctgctgcgcctcgcccacacgctgcgcctcgtgcgcccgcccgtcgtctgggtcgtcgtcgagcccgccgccgacgcgcccgccaccgctgaggtgctccgcggcaccggcgtcatgtacaggcacctcgccttcaagcccgaggacaacttcaccaccgccgacgccgaggctcacgtgcagcggaacgccgcgctcgcgcatgtcgagaagcaccgactcgccggcgtcctgcacttcgccgacgccgccggcgtctacgacgtcggcttcttcgaacagatccgccagatcgagtcagtaccccctcttcccctcccaccaccttcaaatctcgccttcttccttctatttcatgccatgattgtttttaattaatgaaatgactcatgctttcattgatccttcatcttcttacttattaaaatccgtctaggctattaagatgcaaattatttccaagcaagtttcctgatttctttcctatttttggtttccaagctcaaagattggagctttgcctggcccctctttcaatttcacatctaactgatctccatttccagcacatgctgttcttccagaatgtatgttgcttgaatccgaatcacaatgcctaaaattgcaattccagctcataaattccggttctttttttttccccaccgactaggactacgactagggactcctcataactaatcatcgtttcttaattcagtaccataagttattttatttactcgtctaaataaatgctttattagccgtgtagtttggtacataaataaataattaaaaacataagctatcattagcgggggaataagggtgggacaatgtgtgtctcgcatgtacttaccaaactattcttcttaatgctctgaagggggaaaaaagggaggaggcactgtagattctcagagaacttcaactattctctccatttctctgatgcgaggagtcaattattggatctagcttatcactttcctgagtccctttcgcccccaccaaacaatgcttcttttaatctttcgcttttgctcacccccaaggccccaacggacaaatgtgttcatgccaaccaccccaactagtttaatttagactttaaacacgtatcatgagcaaagccacaattattttatgtggtagtggtagcactgctgttagtcactgacaaatgggcctgtaagagtgtcggtggcacctaacagattggaaagtcagtgagaacatgacatccttgaattgactcctgagatgttgttactcctatactggggaagcaacatgctggggggaaatagctacagcctacaaaagatagcaggcagggagaaaaatagtagtaaccgttatgccatggccattgaaactgattgaactaaagtggagagaaggtcctagttctatccacaccattaatttcttcctccaaccccataatgtccatctgatatacaccgcagaaagttgcccacgtattcggtgcagttttcctatactggtgcacacagcaccacctgtgatctagatcttctggagcccgcaagaaactccaaaaagaacttcgtttgcaaaatggcagtatgaatcttcatttctgtgcagaccaccagttgtctttggagatttttctcatgaggagtttggaaagtcttgcaaaaatgagatttcacatttcattttaccaacatttgatgttgattattgggtttcttacttgcatgaccatgaattctcatcagagtatgaaatccagtatttccaccagagcatgacactagtatagcatctgaagtttgttctgaccgcccctttaacccctttgcttatccatctgaacattgttagcggatgctgttacaagtttagatgcttatagatataaagcaatctacatttcaaaaaaaaaaatacatgcataaagcaatcttactcagcatcaaattccgaaaccttttgcctcttaacttgaaaacacatcaacatttgactcaccttttgcattcacttttcccgatggtagcgttctttcattgatgggtgcttgctttcataaagagaggggatcagaccttggtttagtacaatggtactagtagatcactactacagtagtgctcatgccacgtggtagcacagtggcgatcttccctggaagaacagtctttttcgcttagaggaacatgatggggtaaaaacagcctcttttcctcaagcaagattttacctgcacctgggcaccaaaacatctgtgcttgtttttctagtttgccttctcttttttgtgtgtgtgtcccttttaatctttacagtcctcgagatgcgtgcacatctgcctttgtggcccctgatgctttttgtggggctccagcataataatgcaacagaggtaggagcatcaattatgccttgtcgcacgtaggtcatagcatgcctgtcctagaactaccagccaccagaaacaaatgagtgcaggtgaacactacagtgactataagtgtggtttactatactaattggctgggttttgaccgcagttaatgtttcttgcgtctggttgagatttagcattggattcttttaatcctaggaggctgaaatttggttacggtttaccttaatgtttgaataagctcgtttgttccagttgagttttactatacctatgtagttgtgtttgactgcatattgggaacacgtctgcttactcaagtttccattgatcgtgaaccgttgcatcatcccgtagtttgtttgatctggatttgtcaagattattcaaactctggaattggtagtggaacaaattaggcatctgcatgtgcagccgtgcaggcaagtggagggtgtagctgtactgtatagtttacatgcaataactcagtctattccttgtatgaaatgatttggatgagcggtcaaaactatagtagtcagcatgttaaagatgtgtcaatggaggtatcccctcctatcacatgttctttcgagttgaggaaaatcagatgcgtctgtttttttcttccttcgaattaaaatcaattggctgatgtgtggttagtggtgccattctagtcaaggtttggtggcacaatgcttttcaattccataatccataattcttctgacccagtcaagatgagacacatagatcacaggcaaagctacctcttgcgtcacacatgttttggtgcatgttgcatggttcttgttgaatttcatctcagatgttggtgcctttagatgtttttttttctaacatttcaaaatgcgcagggcgtttggtacatggcctgtggcaacaatgtcggcgggcgagaagaaagtggtagtggagggtccactgtgcagtgcctcaaaggtcgtcggttggttctcaagggacttcaacgacgggaccacccgttcagtgacgtataacacggaagctgacctgaatcctgctggtgccgctggtacacgagcccacacgatcgacgtctcaggttttgcgttcaatagctctattttgtgggatcctgagcggtggggacggccaacgtcattaccagacacttcacaggttagtccaaccccgcctttgtggagagagtgttagtgaccgagcaagtctagttcaattaaattttgctttggcatgatacaacatactccctccgattttttttatttgacgtttgttagtataaagttgaactatctcaaacaaaaaatgggggtagtacttagcatttagcaagctatggtatgtggttgactcagtgaagggactggttctcagatattagcaatctttggctgtccatctcttgagaatgtcgtcgtataaaaactctgaattgagattattaatttgaagatgaaatgttaagccattggcattagtaatttgaagtgttatgtcaaggaatggtttcatactctgaattgttttccttctgttacttgaaaaatgaaaggcatcagtttctaatataacagtgtctacgatgtgggctgtgattggcttgtatgtgttacaaggtcctgtaaaatcttcctttttcttgataaaaaacgtgctaaacacaatctcaaagaaaaaaaaaggcaagcggttggaacagattggatgctgaccttgtcaatggttcaagtcaatcactgcaggtcaagcatgtccactcatgttccttctgaacaaggttcgggtatagtagataaattgtctggattcaattcatgatctttttttatctagaataatcactctcaacatatctcatgatctacttgtcatgtgcaagcaataagctaccaacagttagttactgatgtgattgcaggactcgatcaagttcgtgcaagaagtgctacttgaagaccgagctaagctgaaggggatcccctccgactgctcccagatcatggtgtggcagtacagcgtgcccagctcgcgatagagggcagccgagaggacgcacaagaggcgtcgcaacctcgcaggcaggggaagaactgcagggggttggatcggagtcgctgtaaatgtggtgggcgatggtaacggcaaggaaagcaagaaggctgattactaagggttctttttctctctctctcctttttgtgttccctttttctttcccctcagttttagttttcaggtttgggaggggggccatgaatgatgaactgtccgtgggagcaaaagaattgcgatagtatttattcagattcatttgtgattctttagcctggtcggttggtcctgttgtgtttgggttaaaaaaaatggcagtggcgtgaggaatggagaaagtttttattgtgttatggcatgaaatcccagcaaggcagcagcaattaacatctggagaacataaaaatcatgtgcaaagggaaagaaaaaaaagagagaagagactgaatctgcgctcaggtcttgctgtcatcgtccctggtgtttccttgcgggacagtgtttatttgcttttgaaaaaggtctgtgctttgcttgtaagtagagatggcaatgggtacccgaaacccgatgggtaaaaaccctattagggtacgggtatggcgaattttgatacccatgggtattttattgggtcatttgttatgcccatcgggtacggtgggcgtgagtatgttctctgttgccccatacccgctacccgatggggaacccgctaacatatgacacgtgggtcggatttatatgtcccatataaaatgaatttgctctcgttatttgataaccctaagaccctaacgcggccaccagggcaccacgcaccaggcaccaggccagccgccagacggaccatccagaagtcttgtatattcaaatattaagtattgtgtattgaacaattagatttgaagg

SEQ ID NO: 3 - DNA, 1095 bp, Zea mays
atgggcacgggcgcggtggcggtggcggcgccggaccgggccaagcagcggcgggccggcggccacctgtggaagaaggcactgctccacttctccctctgcttcgtcatgggcttcttcacgggcttcgcgccttcctcctcctcctcctggaagtccgccgccgccaccccccagcaccacccgccgcaccgtcccggggaccgcctcgccgcctcccgcgtggccgtcgacgcgcgcgccaccctgtcgccgcccgcggaggccgccgatgcgctctccgccggcgccacggtggacttgggcgacgacgaggaggacgccgcccagcgccgcctcctgatcgtggtcaccaccacgcgctccggccccggggagcgccgccgccgccggggcgagctgctgcgcctcgcccacacgctgcgcctcgtgcgcccgcccgtcgtctgggtcgtcgtcgagcccgccgccgacgcgcccgccaccgctgaggtgctccgcggcaccggcgtcatgtacaggcacctcgccttcaagcccgaggacaacttcaccaccgccgacgccgaggctcacgtgcagcggaacgccgcgctcgcgcatgtcgagaagcaccgactcgccggcgtcctgcacttcgccgacgccgccggcgtctacgacgtcggcttcttcgaacagatccgccagatcgaggcgtttggtacatggcctgtggcaacaatgtcggcgggcgagaagaaagtggtagtggagggtccactgtgcagtgcctcaaaggtcgtcggttggttctcaagggacttcaacgacgggaccacccgttcagtgacgtataacacggaagctgacctgaatcctgctggtgccgctggtacacgagcccacacgatcgacgtctcaggttttgcgttcaatagctctattttgtgggatcctgagcggtggggacggccaacgtcattaccagacacttcacaggactcgatcaagttcgtgcaagaagtgctacttgaagaccgagctaagctgaaggggatcccctccgactgctcccagatcatggtgtggcagtacagcgtgcccagctcgcga

SEQ ID NO: 4 - DNA, 19 bp, Artificial
  synthetic primer
tgagtggacatgcttgacc

SEQ ID NO: 5 - DNA, 20 bp, Artificial
  synthetic primer
cgtataacacggaagctgac

SEQ ID NO: 6 - DNA, 19 bp, Artificial
  synthetic primer
gattgcaggactcgatcaa

SEQ ID NO: 7 - DNA, 20 bp, Artificial
  synthetic primer
tttaacccaaacacaacagg

SEQ ID NO: 8 - DNA, 25 bp, Artificial
  synthetic primer
cttcgtccataatggcaattatctc

(19) United States
(12) Patent Application Publication
(10) Pub. No.: US 2013/0198892 A1
(43) Pub. Date: Aug. 1, 2013

(54) METHOD FOR MODIFYING THE BLOOMING DATE OF A PLANT

(71) Applicants: Jean-Pierre Martinant, Vertaizon (FR); Christophe Tatout, Salt En Donzy (FR)

(72) Inventors: Jean-Pierre Martinant, Vertaizon (FR); Christophe Tatout, Salt En Donzy (FR)

(73) Assignees: GENOPLANTE-VALOR, Paris (FR); BIOGEMMA, Paris (FR)

(21) Appl. No.: 13/635,280

(22) PCT Filed: Mar. 15, 2011
(86) PCT No.: PCT/EP2011/053908

§ 371 Date: Nov. 5, 2012

(30) Foreign Application Priority Data
Mar. 16, 2010 (EP) 10305259.3

(51) Int. Cl. C12N 15/82; A01H 1/02

(52) U.S. Cl. CPC C12N 15/8241; A01H 1/02
USPC 800/275; 44/307; 426/618; 435/320.1; 800/290; 800/298; 800/320.1

(57) ABSTRACT

The present invention applies to the field of plant improvements, in particular to the modification of the blooming date thereof, by manipulation of the Prec31 gene.

The present invention relates to the area of improvement of plants, in particular modification of their flowering date.

The development of plants comprises a vegetative phase and a reproductive phase. Flowering represents the transition between these two phases.

Flowering is thus an essential event in the life cycle of plants and is of great importance for selecting plants that are adapted to their environment.

A plant's flowering date reflects its capacity for adapting to its environment and notably to the climatic conditions, and to external stresses.

Maize has a male organ and a female organ, which bloom asynchronously.

A great number of environmental signals (such as length of day, temperature (vernalization), availability of nutrients, of water or of phytohormones) control the transition between the vegetative and reproductive phases, and may therefore influence the flowering date.

The flowering date of maize is also influenced by various genetic factors. Thus, Buckler et al. (Science, 2009, 325, p. 714-718) describe the identification of chromosomal regions of maize (QTL, for Quantitative Trait Loci) influencing the flowering of these plants. These regions were identified using a large population of plants. The authors pointed out that the differences observed between consanguineous lines are not caused by a few genes having a large effect, but rather by the cumulative effect of several QTL. Thus, the QTL identified rather display minor effects (bringing flowering forward by at most 1.5 days).

In fact, there are at least 80 flowering genes in Arabidopsis (see Blazquez et al. 2001 (EMBO Rep. 2001 December; 2(12):1078-82). The number of orthologs of these genes in maize is very large: thus, Buckler et al. talk of 1000 flowering genes of Arabidopsis having homologs in maize.

Identification of genes involved in flowering in other species has been described in various documents. We may notably mention WO 2006/068432, which describes the genes OsMADS50, OsMADS51, OsMADS56, OsMADS14 (truncated), OsTRXI, OsVIN2, OsCOL4, and OsCOL8. These various genes show an effect on rice, an autogamous plant. The inventors of the previous application then studied in more detail the role of OsMADS51 (Kim et al., Plant Physiol. 2007 December; 145(4): 1484-94), confirming the effects reported in WO 2006/068432, and specifying a genetic sequence (architecture) of flowering in rice.

The inventors have identified a gene coding for a protein that will be designated Prec31 in the present application, and whose amino acid sequence is represented by SEQ ID No. 1.

In maize, a mutant in the gene coding for this protein has an early male and female flowering date relative to the quasi-isogenic nonmutant plant.

The present invention thus relates to a method for bringing forward the flowering date of a plant, characterized in that the expression and/or activity, in said plant, of a protein designated Prec31, whose polypeptide sequence has at least 75% identity with sequence SEQ ID No. 1, is inhibited totally or partially.

In the context of the present description, the elements compared (bringing forward of the flowering date, earlier or later flowering date) are compared relative to quasi-isogenic plants, except with regard to the object of the invention (inhibition of Prec31, presence of a mutation or of a transgene intended to inhibit Prec31). A quasi-isogenic plant possesses a genome at least 95% identical to the genome of the plant according to the invention, except with regard to the presence of elements inhibiting Prec31. A quasi-isogenic line can be obtained by backcrossing of the plant according to the invention with the line with which one wishes to establish the comparison.

The principle of this is recalled hereunder: A series of backcrosses is carried out between the line with which one wishes to effect a comparison and the line bearing the element(s) inhibiting Prec31.

In the course of the backcrosses, one can select the individuals bearing an element or elements inhibiting Prec31, and having recombined the smallest fragment of the donor line around this transgene. In fact, by means of molecular markers, individuals are selected that have, for the markers near the gene, the genotype of the elite line.

Moreover, it is also possible to accelerate the return to the elite parent owing to molecular markers distributed on the whole genome. At each backcrossing, the individuals having the most fragments from the recurrent elite parent will be selected.

With good implementation, starting from the fourth generation, it is possible to obtain a quasi-isogenic line of the elite line, i.e. identical to the initial elite line, but which has integrated the element(s) inhibiting Prec31. The fragment integrated bears the element(s) inhibiting Prec31, as well as any flanking chromosomal regions derived from the initial maize (notably the well-known lines A188 or Hi-II, used for transformation when evaluating the effect of a transgene in maize).

Said plant is notably maize, rice, sorghum, millet, wheat and other straw-type cereals, or a fodder plant.

Protein Prec31 is defined herein as any protein whose polypeptide sequence has at least 75%, preferably at least 85%, or at least 90%, advantageously at least 94%, and quite preferably at least 95%, more preferably at least 97% identity with sequence SEQ ID No. 1 on a comparison window that is as wide as possible, preferably corresponding to the whole of sequence SEQ ID No. 1. Unless stated otherwise, the percentages of identity stated here are established by means of the BLAST2 software (ALTSCHUL et al., Nucleic Acids Res., 25, 3389-3402, 1997) using the default parameters (BLOSUM62 matrix for comparisons of proteins, with penalties of 11 for an open gap, 1 for an extension gap, 50 for a “gap x_dropoff”, a random wait value of 10, a word size of 3, and no filter).

Total or partial inhibition of the expression and/or activity of protein Prec31 can be obtained in various ways, known by a person skilled in the art.

Preferably, expression of the gene coding for Prec31 is decreased by mutagenesis, or else by inhibition or modification of its transcription or of its translation.

The Prec31 gene is understood as comprising the coding and noncoding regions (untranslated 5′ or 3′ regions, introns) of the gene.

Mutagenesis of the gene coding for Prec31 can occur at the level of the sequence encoding or of the sequences regulating the expression, notably of the promoter, or of the untranslated 3′ region (3′UTR). For example, some or all of said gene can be deleted and/or an exogenous sequence can be inserted. As an example, one may mention insertional mutagenesis: a large number of individuals is produced deriving from a plant with activity for transposition of a transposable element (AC or Mutator element in maize), and the plants in which an insertion has been effected in the gene of Prec31, or in proximity with an effect on translation or expression, are selected, for example by PCR.

Any other method known by a person skilled in the art for inhibiting the gene coding for Prec31 can also be used. Thus, mutation of the genes can be effected by inserting a transposable element or a transfer DNA (tDNA). Physical or chemical mutagenesis can also be carried out, notably using EMS, X-rays or ultraviolet radiation (see notably McCallum et al., Plant Physiol., 123, 439-442, 2000). The mutations of interest lead to shifting of the reading frame and/or introduction of a stop codon into the sequence and/or modification of the level of transcription and/or translation of the gene and/or making the enzyme less active than the wild-type protein.

The plants thus mutated are screened for example by PCR, using primers located in the target gene. However, other methods of screening can also be used, such as Southern blotting or screening using the AIMS method described in WO 99/27085 (for detecting insertions), using probes specific to the target genes, or methods of detection of point mutations or of small insertions/deletions using particular endonucleases (Cel I, Endo I) as described in WO 2006/010646.

In another embodiment, inhibition is obtained by transformation of the plant with a vector containing a sense or antisense construct of the target gene. These two methods are known to permit, in certain conditions, inhibition of the target gene. The RNA interference (RNAi) method is also used, and is particularly effective for the extinction of genes in plants. This method is well known by a person skilled in the art and consists of transformation of the plant with a construct producing, after transcription, a double-stranded RNA duplex, one of the strands of which is complementary to the mRNA of the target gene (for a review of post-transcriptional inhibition techniques: Chicas and Macino, EMBO Reports, 21(11), 992-996, 2001; for a review of the use of interfering RNAs: Hannon, Nature, 418, 244-251, 2002).

The present invention thus notably relates to the use of at least one polynucleotide selected from:
a) a polynucleotide coding for a protein Prec31 as defined above;
b) a polynucleotide complementary to a polynucleotide a) as above;
c) a fragment of at least 12 consecutive nucleotides, of a polynucleotide a) or b) as above, or capable of hybridizing selectively to said polynucleotide;
d) a polynucleotide permitting the production of a duplex usable for RNA interference (RNAi), said one polynucleotide containing a polynucleotide X containing at least 12 nucleotides, preferably at least 15, advantageously at least 20, and quite preferably at least 50 nucleotides capable of hybridizing selectively to a polynucleotide defined in a), as well as a polynucleotide Y complementary to said polynucleotide X
   for obtaining a plant having an earlier flowering date.

A polynucleotide coding for Prec31 is a polynucleotide containing the genetic information permitting synthesis of the protein Prec31 represented by SEQ ID No. 1, or of a protein having at least 75%, preferably at least 85%, or at least 90%, advantageously at least 94%, and quite preferably at least 95%, more preferably at least 97% identity with SEQ ID No. 1.

This notably encompasses the genomic DNA whose sequence SEQ ID No. 2 given in the appendix represents an allele, as well as the corresponding cDNA (SEQ ID No. 3). The coding portion comprises the nucleotides 979-1674, 4667-4948 and 5712-5831 (including the STOP codon) of SEQ ID No. 2.

A fragment specific to a polynucleotide a) or b) above is a fragment of said polynucleotide whose sequence is not found in other genes of the same plant.

A polynucleotide capable of hybridizing selectively to a polynucleotide a) or b) as above is a polynucleotide which, when it is hybridized in stringent conditions to a nucleic acid bank from the same plant (notably a bank of genomic DNA, or of cDNA), produces a detectable hybridization signal at least 2 times greater, and preferably at least 5 times greater than the background noise with said polynucleotide, but does not produce any detectable signal with other sequences of said bank.

A person skilled in the art is able to define the stringency of the hybridization conditions, which depend on the size and the composition of bases of the polynucleotide in question, as well as the composition of the hybridization mixture (notably pH and ionic strength). Generally, stringent conditions, for a polynucleotide of a given size and sequence, are obtained by working at a temperature about 5° C. to 10° C. lower than the melting point (Tm) of the hybrid formed, in the same reaction mixture, by this polynucleotide and its complement.

The present invention relates in particular to a method for bringing forward the flowering date of a plant, by total or partial inhibition of the endogenous protein Prec31 of said plant, comprising the transformation of said plant with a recombinant DNA construct comprising a polynucleotide as defined above, positioned in sense orientation or in antisense orientation, or that can be transcribed to double-stranded RNA, under the transcriptional control of a suitable promoter.

The invention also relates to a method for delaying the flowering date of a plant, by overexpression of a protein Prec31 in said plant. This overexpression is achieved by transformation of said plant with a recombinant DNA construct comprising a polynucleotide as defined above under the transcriptional control of a suitable promoter. The transgenic plants thus obtained are also an object of the invention.

The present invention also relates to recombinant DNA constructs comprising a polynucleotide as defined above. These constructs are notably expression cassettes, comprising a polynucleotide as defined above, under the transcriptional control of a suitable promoter. These expression cassettes can also contain other regulatory elements, in particular regulatory elements of transcription such as terminators and/or amplifiers.

Recombinant vectors that comprise polynucleotides as defined according to the invention or an expression cassette according to the invention are also objects of the invention.

Said vectors can also comprise other elements, for example one or more selection markers, and can notably be used for obtaining transgenic plants.

As nonlimiting examples of promoters usable in the context of the present invention, we may mention constitutive promoters, such as the 35S promoter of the cauliflower mosaic virus (CaMV), or derivatives thereof, the promoter of the cassava vein mosaic virus (CsVMV) (WO 97/48819), the promoter of ubiquitin or the actin-intron-actin promoter, of rice (McElroy et al., Mol. Gen. Genet., 231, 150-160, 1991; GenBank S 44221).

Inducible or tissue-specific promoters can also be used. This makes it possible for inhibition of Prec31 only to be induced at certain stages of development of the plant, in certain environmental conditions, or in certain target tissues, for example stems, leaves, seeds, spathes, cortex or xylem.

Advantageously, other regulatory elements of transcription are also used, such as terminators, and notably the 3′NOS terminator of nopaline synthase (Depicker et al., J. Mol. Appl. Genet., 1, 561-573, 1982), or the 3′CaMV terminator (Franck et al. Cell, 21, 285-294, 1980; GenBank V00141).

The selection marker genes usable in the context of the present invention are notably genes conferring resistance to an antibiotic such as hygromycin, kanamycin, bleomycin or streptomycin, or to a herbicide (EP 0 242 246) such as glufosinate, glyphosate or bromoxynil. The nptII gene that confers resistance to kanamycin can thus be used.

The transformation of plants can be carried out by many methods that are known per se by a person skilled in the art.

It is possible, for example, to transform plant cells, protoplasts or explants, and regenerate a whole plant from the material transformed. Transformation can thus be performed by transferring vectors according to the invention into protoplasts, notably after incubation of the latter in a solution of polyethylene glycol (PG) in the presence of divalent cations (Ca2+) according to the method described in the article by Krens et al. (Nature, 296, 72-74, 1982) or by electroporation notably according to the method described in the article by Fromm et al. (Nature, 319, 791-793, 1986). It is also possible to use a gene gun for propelling, at very high speed, metal particles coated with the DNA sequences of interest, thus delivering genes into the cell nucleus, notably by the technique described in the article by Finer et al. (Plant Cell Report, 11, 323-328, 1992) or cytoplasmic or nuclear micro-injection.

The method of transformation by Agrobacterium tumefaciens will preferably be used, notably according to the method described by Ishida et al. (1996, Nature Biotechnol. 14, 745-50).

The present invention also relates to the plant cells and the transgenic or mutant plants obtainable by a method according to the invention, and the cells and plants containing a recombinant polynucleotide or an expression cassette as defined above. The invention also relates to the plant cells and the plants obtained after mutation of the gene coding for Prec31 in such a way that it leads to inhibition of the expression and/or activity of the protein.

Of course, the present invention includes the plants derived from plants according to the invention, which are notably descendants, notably hybrids resulting from crossing involving at least one plant according to the invention, obtained by sowing or by vegetative propagation of the plants according to the invention or obtained directly or indirectly by a method according to the invention.

The invention also comprises the plant cells and tissues, as well as the organs or parts of plants, including leaves, stems, roots, flowers, fruits, and/or seeds obtained from a plant according to the invention.

Preferably, the invention applies to maize and to cells and tissues derived from maize.

The invention relates in particular to maize or a maize seed having an allele of the Prec31 gene, called delta-Prec31 comprising an insertion of a transposon in the untranslated 3′ region, 297 nucleotides after the stop codon of the translated region, said allele being present in a representative sample of seeds deposited at the NCIMB under number NCIMB 41706.

Seeds possessing the delta-Prec31 allele were deposited at NCIMB Limited, Ferguson Building, Craibstone Estate, Bucksburn, Aberdeen, Scotland, AB21 9YA, UK, on Mar. 15, 2010, in accordance with the provisions of the Budapest Treaty, under number NCIMB 41706.

The present invention also relates to a method of selecting maize, or to a method of identifying a maize usable in a selection (breeding) scheme intended for obtaining a maize having an early flowering date (earlier than the average flowering date of the maizes used in the selection (breeding) scheme), characterized in that it comprises searching for an allele of the Prec31 gene possessing a mutation resulting in an earlier flowering date of said maize, relative to a maize not possessing said allele. This method is directly connected with the demonstration of the role of Prec31 in bringing forward the flowering date.

In a preferred case, said mutation results in total or partial inhibition of the expression and/or activity of Prec31.

Said allele can be found by direct detection of the mutation responsible for inhibition; it can also be found by detection of the allelic form associated with this mutation of a polymorphism with linkage disequilibrium with the latter.

These mutant alleles thus identified can then be introgressed into selected lines, and notably into elite lines, namely lines having high agronomic and commercial potential.

In a particular embodiment, a polymorphism localized at nucleotide 5089 of SEQ ID No. 2 is sought. When the nucleotide is a T, flowering is earlier. When the polymorphism is a deletion, flowering is delayed.

In a particular embodiment, a polymorphism localized at nucleotide 5025 of SEQ ID No. 2 is sought. When the nucleotide is a G, flowering is earlier. When the nucleotide is a T, flowering is delayed.

In a particular embodiment, a polymorphism localized at nucleotide 4961 of SEQ ID No. 2 is sought. When the nucleotide is an A, flowering is earlier. When the nucleotide is a C, flowering is delayed.

In a particular embodiment, the presence of two polymorphisms at nucleotides 5089 and 5025 of SEQ ID No. 2 is sought. In another embodiment, the presence of two polymorphisms at nucleotides 5089 and 4961 of SEQ ID No. 2 is sought. In another embodiment, the presence of two polymorphisms at nucleotides 5025 and 4961 of SEQ ID No. 2 is sought.

In another embodiment, the presence of three polymorphisms at nucleotides 4961, 5025 and 5089 of SEQ ID No. 2 is sought.

It should be noted that the presence of two haplotypes A/G/T, and C/T/- is often observed, corresponding to an early allele for the first, and a late allele for the second.

Preferably, the transgenic maize according to the invention is an “elite” maize. A person skilled in the art is familiar with the definition of an elite maize. It is a maize intended for generating hybrids that are intended to be commercialized by crossing with another elite maize. An elite maize is defined as such in relation to the territory envisaged for marketing, as well as the agronomic character(s) required for the hybrid offspring. Notably it is a maize that can be included in a reference catalog.

Thus, if the offspring is intended for animal feed, the characters of yield (to find a regular, high yield in tonnage of dry matter per hectare), of ingestibility and of digestibility will be evaluated, when assessing the “elite nature of maize”. If the offspring is intended for production of biofuels or biogas, maizes having the best energy yield after transformation will be evaluated.

In order to determine the elite character of a maize, hybrids obtained from the latter are compared with the reference commercial hybrids (sold for the same objective in the same region), by field trials, by recording and measuring agronomic characters appropriate to the required objective. A maize is defined as elite if the results obtained for the parameters investigated for a hybrid obtained by crossing of said maize are 90% higher than the results recorded for the same parameters of the reference hybrids.

Thus, an elite maize is a maize combining the maximum of agronomic characteristics necessary for economic penetration of the intended market. As today's market for maize is a market of hybrids, the elite character of a maize is also evaluated by the suitability of said maize for combination/production of hybrids.

Thus, application of the present invention makes it possible to obtain an elite maize intended for the commercialization of hybrids for any use, said maize displaying early flowering. This elite maize is therefore homozygous for the delta-Prec31 allele.

In another embodiment, the invention makes it possible to obtain a hybrid maize obtained by crossing two homozygous parent lines, said hybrid maize having a delta-Prec31 allele. This hybrid maize can be homozygous (if each homozygous parent has the delta-Prec31 allele) or heterozygous for the allele.

Any plant as described above (transgenic or mutant) can contain one or more transgenes in addition to elements inhibiting Prec31 and the delta-Prec31 allele. One may mention transgenes conferring male sterility, male fertility, resistance to a herbicide (notably glyphosate, glufosinate, imidazolinone, sulfonylurea, L-phosphinotricine, triazine, benzonitrile), resistance to insects (notably a transgene coding for a Bacillus thuringiensis toxin), tolerance to water stress. These plants can be obtained by crossing said plants of the invention with other plants containing said transgenes. Alternatively, plants can be co-transformed with an expression cassette containing several different transgenes, including those inhibiting Prec31.

The invention relates in particular to a method for obtaining a maize having an early flowering date, comprising introgression of the delta-Prec31 allele in said maize, comprising the steps consisting of:
a) crossing a first line of maize having the delta-Prec31 allele with a second maize that does not have said allele,
b) carrying out genotyping of the offspring obtained and selecting the descendants having the delta-Prec31 allele, and optionally having the best genome ratio for said second maize,
c) carrying out backcrossing of said descendants with said second line of elite maize usable for the production of hybrids,
d) repeating if necessary steps b) and c) until an isogenic line of said second maize, having the delta-Prec31 allele, is obtained,
e) optionally, carrying out self-fertilization in order to obtain a plant that is homozygous for the delta-Prec31 allele.

When the line of maize having the delta-Prec31 allele is homozygous for this allele, there is no need to carry out the genotyping envisaged in step b) after the first crossing envisaged in step a).

Thus, the teachings can be used for identifying maizes usable in a selection (breeding) scheme intended for selecting maizes having an earlier flowering date than the average flowering date of the maizes used in the selection scheme, by examining for the presence of the delta-Prec31 allele.

Finally, the invention relates to the use of a plant, and notably of a maize according to the invention for preparing a composition intended for human or animal nutrition, or for preparing biofuels or for any other industrial application, as well as to the methods using said plants for said applications.

### EXAMPLES

### Example 1

### Characterization of the Delta-Prec31 Mutant

A line of maize having an insertion of a transposable element after the G located in position 6125 of the reference sequence SEQ ID No. 2 is isolated. The allele thus obtained is called delta-Prec31.

Although present in the untranslated 3′ region of the gene, it is assumed that this insertion leads to deregulation of the transcription and/or translation of the Prec31 gene, or of the stability of the mRNA of Prec31, leading to a decrease in the activity of the protein in the presence of the delta-Prec31 allele.

For more precise investigation of the effect of the insertion observed in the Prec31 gene in an elite maize, successive backcrosses are carried out with an elite line of maize.

This method permits very rapid production of quasi-isogenic lines only differing by the locus bearing the modified allele, the descendants being tested for possessing a genome ratio as close as possible to that of the elite parent while having the allele that we wish to introgress. These tests are assisted by molecular markers (techniques that are well known, microsatellites, AFLP, etc.). For assessing the effect of the insertion as soon as possible (production of homozygous plants), self-fertilizations are carried out at the various intermediate stages of backcrossing.

In this way, early flowering is evaluated on the BC3S1 plants (3 backcrosses in one elite line, 1 self-fertilization).

The following results are observed:

Male flowering (MF): Flowering significantly earlier at the mutant homozygous state relative to the quasi-isogenic wild-type homozygote. The effect is very clear: P value=0.0046 and significant difference of 5 days between the average flowering dates of the mutant homozygotes compared to the average flowering dates of the wild-type homozygotes.
Female flowering (FF): Flowering also significantly earlier at the mutant homozygous state relative to the quasi-isogenic wild-type homozygote. P value=0.0221 and significant difference of 4 days between the average flowering dates of the mutant homozygotes compared to the average flowering dates of the wild-type homozygotes.

The mutation therefore causes flowering to be earlier by around 4 to 5 days, both for male flowering and for female flowering.

In order to determine whether the insertion is in homozygous or heterozygous form, the following primer pair can be used:

SEQ ID No. 6: GATTGCAGGACTCGATCAA, upstream of the insertion, and a primer of sequence SEQ ID No. 7: TTTAACCCAAACACAACAGG, downstream of the insertion.

In addition to these two primers, the OMuA primer SEQ ID No. 8 CTTCGTCCATAATGGCAATTATCTC specific to the endogenous transposable element is used. This primer is directed toward the “exterior” of the transposon. These three primers can be used simultaneously in a PCR amplification experiment from genomic DNA (hybridization temperature=58° C.). Deposition of the amplification products on gel reveals:
obtaining a single band about 450 bp long for so-called wild-type plants at this locus (i.e. not having the mutation);
obtaining a band of the order of 500 bp for mutant homozygous plants
or obtaining two bands for heterozygous plants.

### Example 2

### Searching for Other Mutations in the Prec31 Gene

The primers TGAGTGGACATGCTTGACC (SEQ ID No. 4) and CGTATAACACGGAAGCTGAC (SEQ ID No. 5) are used for amplifying a region straddling the end of the second exon and the start of the second intron of the Prec31 gene.

These primers amplify a region of 754 nucleotides from nucleotide 4803 to nucleotide 5556 of SEQ ID No. 2.

The presence of 3 polymorphisms is observed, located at 4961, 5025 and 5089 of SEQ ID No. 2.

These nucleotides have the following effect on the flowering date:

| Polymorphism | Allele 1 | Effect | Allele 2 | Effect |
|---|---|---|---|---|
| 4961 | C | 25.94 | A | −25.94 |
| 5025 | T | 25.77 | G | −25.77 |
| 5089 | — | 25.57 | T | −25.57 |

The effects are shown in degree-days. In fact, when there is large interannual variability of temperatures, the duration of the development cycles is not evaluated in number of days, but in total degree-days. Put simply, the total degree-days corresponds to the sum of the daily mean temperatures. In the present case, as the temperature observed during flowering is of the order of 20° C., the polymorphisms bring forward or delay the flowering date by just over one day.

The comparison is made relative to the mean value of the flowering dates for all of the lines of a panel of 374 lines of maize representing a wide genetic diversity. The dates adopted for each of the polymorphisms correspond to the mean value of the flowering dates for all the lines containing said polymorphism.

Owing to the great variability between the lines used, this method makes it possible to minimize the effect of the other genetic loci that may be involved in flowering.

The SNP most associated with earlier flowering is located in position 4961 of the reference sequence SEQ ID No. 2. It is very significant (associated with a p value of 5.1×10⁻⁴) and has an early flowering allele A of 25.94° days and a delaying allele C of 25.94° days. The percentage phenotypic variance associated with this locus is 5%.

It should be noted that the reference sequence SEQ ID No. 2 supplied for locating the polymorphisms of the Prec31 gene possesses the polymorphisms of the haplotype 2, and is therefore associated with early flowering.

These results confirm the involvement of the Prec31 gene in the mechanism of flowering.

### Example 3

### Expression of Prec31

Tissue samples (apex, internal part of the sheath and external part of the sheath) were collected at different foliar stages and notably at the 6.3 leaves stage, i.e. the floral transition stage (flowering “switch”) which corresponds to the stage of transition from the vegetative stage to the floral stage.

It could be shown that Prec31 has a differential transcriptional expression between the internal part of the sheath and the external part exposed to the light. At the 6.3 leaves stage, the Prec31 gene is more strongly expressed in the external part.

This expression profile is the inverse of that of the id1 (INDETERMINATE 1) gene in maize (Colasanti et al., Cell, May 15, 3(4): 593-603, 1998) known to induce flowering.

These results, added to the fact that the Prec31 gene is colocalized with an early flowering QTL located on chromosome 8 (QTL at bin 8.05), confirm the involvement of the Prec31 gene in the mechanism of flowering.

1. A method for bringing forward the flowering date of a plant, comprising totally or partially inhibiting the expression and/or activity in said plant of a protein designated Prec31, whose polypeptide sequence has at least 75% identity with the sequence SEQ ID No. 1.

2. The method as claimed in claim 1, characterized in that said plant is maize.

3. The method of claim 1, characterized in that at least one polynucleotide selected from the group consisting of
a) a polynucleotide coding for a protein Prec31 as defined in claim 1;
b) a polynucleotide complementary to a polynucleotide a) as above;
c) a fragment of at least 12 consecutive nucleotides, of a polynucleotide a) or b) as above, or capable of hybridizing selectively to said polynucleotide;
d) a polynucleotide permitting the production of a duplex usable for RNA interference (RNAi), said one polynucleotide containing a polynucleotide X containing at least 12 nucleotides, preferably at least 15, advantageously at least 20, and quite preferably at least 50 nucleotides capable of hybridizing selectively to a polynucleotide defined in a), as well as a polynucleotide Y complementary to said polynucleotide X
is used to inhibit the expression and/or activity in said plant of the protein designated Prec31.

4. The method as claimed in claim 1, characterized in that inhibition of the expression and/or activity of the enzyme Prec31 is obtained by mutagenesis of the gene coding for said enzyme, said gene comprising the coding and noncoding regions (5′, 3′, introns) of the gene.

5. The method as claimed in claim 1, characterized in that it comprises transforming said plant with a recombinant DNA construct comprising a polynucleotide as defined in claim 3, under the transcriptional control of a suitable promoter.

6. An expression cassette comprising a polynucleotide as defined in claim 3, under the transcriptional control of a suitable promoter.

7. A recombinant vector containing an expression cassette as claimed in claim 6.

8. A plant obtainable by the method as claimed in claim 4, and having a mutation in the gene coding for protein Prec31 whose sequence has at least 75% identity with SEQ ID No. 1, said mutation resulting in inhibition of the expression and/or activity of Prec31.

9. A genetically modified plant, obtainable by a method as claimed in claim 5.

10. The plant as claimed in claim 8, characterized in that it is a maize.

11. A method of selecting maize, characterized in that it comprises searching for an allele of the gene of protein Prec31 possessing a mutation resulting in an earlier flowering date of said maize relative to a maize not possessing this allele.

12. The method as claimed in claim 11, characterized in that said allele is represented by SEQ ID No. 2.

13. A maize or maize seed having an allele of the Prec31 gene, called delta-Prec31, comprising an insertion of a transposon in the untranslated 3′ region of the Prec31 gene, said insertion being localized after the G located in position 6125 of the reference sequence SEQ ID No. 2, said allele being present in a representative sample of seeds deposited at NCIMB under number NCIMB 41706.

14. (canceled)

15. A method for obtaining a maize having an early flowering date, comprising the introgression of the delta-Prec31 allele in said maize, comprising:
a) crossing a first line of maize having the delta-Prec31 allele with a second maize that does not have said allele,
b) carrying out genotyping of the offspring obtained and selecting the descendants having the delta-Prec31 allele, and optionally having the best genome ratio for said second maize,
c) carrying out backcrossing of said descendants with said second line of elite maize usable for the production of hybrids,
d) repeating if necessary steps b) and c) until an isogenic line of said second maize, having the delta-Prec31 allele, is obtained,
e) optionally, carrying out self-fertilization in order to obtain a plant that is homozygous for the delta-Prec31 allele.

16. The use of a maize as claimed in claim 10, or obtained according to the method of claim 15 for preparing a composition intended for human or animal nutrition, or for preparing biofuels or for any other industrial application.

17. The maize or maize seed of claim 13, wherein said maize or maize seed is a maize.

18. The maize or maize seed of claim 13, wherein said maize or maize seed is a maize seed.
